(19) United States
(12) Patent Application Publication
(10) Pub. No.: US 2023/0380838 A1
(43) Pub. Date: Nov. 30, 2023

(54) IMPLANTABLE DEVICE FOR REPAIRING AT LEAST ONE TENDON OR LIGAMENT

(71) Applicants: COUSIN BIOTECH, WERVICQ SUD (FR); UNIVERSITEIT GENT, GENT (BE)

(72) Inventors: Ian PEETERS, Brasschaat (BE); Lieven DE WILDE, Gent (BE); Alexander VAN TONGEL, Melle (BE); Stéphane NOEL, Hantay (FR)

(73) Assignees: COUSIN BIOTECH, WERVICQ SUD (FR); UNIVERSITEIT GENT, GENT (BE)

(21) Appl. No.: 18/032,796

(22) PCT Filed: Oct. 7, 2021
(86) PCT No.: PCT/EP2021/077675

§ 371 Date: Apr. 19, 2023

(30) Foreign Application Priority Data
Oct. 22, 2020 (FR) FR2010835

(51) Int. Cl. A61B 17/11

(52) U.S. Cl. CPC A61B 17/1146

(57) ABSTRACT

The present invention relates to an implantable device (10) for repairing at least one tendon or ligament, advantageously comprising a hollow braid (20) configured to receive in its interior volume (30) at least one portion of a tendon or ligament, and comprising over its length a central repair part (40) arranged between first and second lateral parts (50, 60). The braiding angle (β) in at least one region (42) of the central part (40) at rest is smaller than the braiding angle (α1) in at least one region (52) of the first lateral part (50) and/or than the braiding angle (α2) in at least one region (62) of the second lateral part (60) at rest.

### TECHNICAL FIELD

The present invention relates to an implantable device for repairing at least one damaged/ruptured or cut tendon or ligament of the human body or of an animal, preferably of the human body.

### STATE OF THE ART

Following a trauma (handling of a sharp object, during a sports activity, a road accident, etc.), a tendon or ligament can be partially damaged, ruptured or cut, its repair then generally requires numerous surgical interventions. The repair consists in making more or less specific sutures depending on the location and dimensions of the tendon. When a tendon is damaged or cut, it is necessary to join and assemble the edges or cut portions or reconsolidate the damaged portions.

Particularly, the tendons, in particular flexor tendons, for example of the hand, are arranged in fibrous sheaths, which sheaths are anchored to a bone. These sheaths are hollow and receive portions of a tendon sliding therein. These fibrous sheaths ensure that the tendon is correctly placed upon movement of a joint. These sheaths comprise fibrous segments performing the function of pulleys.

The repaired area of the tendon has the disadvantage of often being swollen, bulky, and of creating adhesions, fibroses, which hinders its sliding in the fibrous sheath.

The repair of the tendons or ligaments using sutures alone has many disadvantages. The rupture of the suture is the most brutal complication, which can occur within 30 days following the repair, statistically it can occur in 5% of the cases. The post-operative infection is the most serious complication, as it will lead to necrosis of the tendinous ends and to a stiffness which is difficult to repair. The development of adhesions limits the sliding of the tendons. Adhesions around the suture will penalize the result and may lead to the need for tenolysis, which is a complex operation and which does not necessarily give a good result despite the skills of the operator. The rehabilitation is longer if it is done following an immobilization of two or three weeks after surgery. The tendon sheath may also over-widen.

The sutures that are the most difficult to repair are located at the base of the fingers of the hand. At this level, the two flexor tendons are interwoven inside the sheath which is particularly narrow. This region is called area 2 of the flexor tendons (cf. FIG. 1 appended to the present application).

Finally, the time for making the sutures is important. In addition, it has been reported that an immediate active or passive mobilization makes it possible to avoid the formation of adhesion and to improve the range of the possible movements. The rehabilitation thus poses a cruel dilemma: 1/ mobilizing early to avoid the stiffening and the adhesions but taking the risk of a new rupture involving a new intervention, or 2/ mobilizing later to avoid the rupture but taking the risk of a significant stiffening and therefore of a re-intervention for tenolysis.

There is thus a need to overcome the various problems relating to tendon repair by suture, making it possible to set up the rehabilitation quickly without risk of rupture of the repair suture between the two ruptured tendons and without damaging the tendon sheaths.

US 2013/0013065 A1 proposes for this purpose a braid receiving in its interior volume the tendons to be repaired. Under the effect of the tensile forces exerted naturally on the ends of the tendons in connection with the ends of the braid, the braid elongates uniformly throughout its length so that the tendons in connection are pinched uniformly over the entire length of the braid. The tendons to be repaired are held in the braid passing through one or more fibrous sheath(s), which facilitates the displacement of the tendons in said sheaths, and thus the activation of an early mobility.

However, the damaged or cut ends of the tendons tend to move apart under the effect of the tensile forces applied to the ends of the braid and therefore correlatively to the tendons, or even to separate. The repair is therefore not ensured correctly. In addition, if there is too great a gap between the two tendon portions to be repaired during healing, the tendon may lose mechanical strength in the long term.

There is thus a need for a device for repairing at least one tendon or at least one damaged or cut/ruptured ligament allowing reliable consolidation and repair while allowing exerting the mobility of the repair device and therefore of the targeted tendons post-operatively, particularly to avoid or limit other surgical interventions.

### DISCLOSURE OF THE INVENTION

The present invention relates to an implantable device for repairing at least one tendon or ligament, advantageously comprising a hollow braid configured to receive in its interior volume at least one portion of a tendon or ligament, and comprising over its length a central repair part arranged between first and second lateral parts. The braiding angle in at least one region of the central part at rest is smaller than the braiding angle in at least one region of the first lateral part at rest and/or smaller than the braiding angle in at least one region of the second lateral part at rest. Advantageously, the mechanical behavior of the central part is differentiated from that of the first and second lateral parts. The first and second lateral parts exert frictional and compressive forces on the tendon they receive that are greater than those exerted by the central part at the start of the installation.

It is thus possible to arrange two tendon portions which partially overlap or are simply juxtaposed in the interior volume of the central part, each portion being extended by a tendon section. A first tendon section is arranged in the interior volume of the first lateral part while the second tendon section is arranged in the interior volume of the second lateral part. Sutures are made to hold the first and second tendon sections inside the first and second lateral parts. One or more suture(s) can be made to keep the tendon portions joined and arranged inside the central part or said joined tendon portions can be freely arranged inside the central part of the braid without suture.

The lower braiding angle in the central repair part gives the latter an improved ability to expand which is higher than that of the first and second lateral parts. This arrangement allows the central part to receive in its interior volume overlapping tendon portions, which superimposed portions create a bulky repair area deforming the central part. The central part thus adopts a domed, ovoid shape. The length of the superimposed portions can for example range from 3 to 4 mm.

In operation, once implanted, the load exerted by the muscles on the tendons, in particular during the rehabilitation, is transferred to the braid, which is tensioned. Particularly, the first and second ends of the braid are subjected to longitudinal tractions, which can be opposite. This longitudinal tensioning leads to a reduction in the diameters of each of the parts of the braid. The elongation of the central part thus allows the overlapping tendon portions to glide relative to each other very gradually while remaining in contact, and thus at the end of the healing to be substantially juxtaposed. Correlatively, the first and second lateral parts contract faster and more strongly than the central part, and adhere by friction to the tendon sections they receive, thus holding the tendon sections in position in the interior volume of the braid. Only the tendon portions be repaired, which are particularly superimposed, are thus allowed to move within the interior volume of the braid. At the end of this process, the tendon portions treated in the central part are substantially butted and assembled together by fusion.

As a non-limiting example, the practitioners now consider that when the tendon portions to be repaired are juxtaposed, they should not be more than 2 mm apart at the start of the healing. Beyond this spacing, the fusion would no longer be possible. The central repair part elongates more than the first and second lateral parts, so that the joined tendon portions are allowed to move gradually until being substantially butted while allowing their gradual assembly.

This differentiated behavior between the central repair part and the first and second lateral parts prevents the joined portions of the tendon from disassembling while firmly holding the first and second ends of the tendon via the first and second lateral parts. The first and second lateral parts thus develop more friction with the tendon in order to adhere to the latter.

Preferably, a braided strand extending continuously between the first and second free ends of the braid has a helical trajectory centered on the longitudinal axis of the braid passing through its center. The helix in the central part thus has a pitch greater than the pitch of the helix in the first lateral part and/or than the pitch of the helix in the second lateral part.

Preferably, the pitch of a helix is the distance of advance of the helix in one complete turn, i.e. 360°.

Furthermore, damaged or cut/ruptured portions of different sizes or complex to suture can be treated with the device according to the invention, thus including the degenerated tendons.

Finally, the repair area comprising the damaged or cut portions of the tendon being held in the central part of the braid, this avoids adhesions with the fibrous sheath(s). The implantable device according to the invention thus facilitates the rehabilitation because the adhesions complicate the rehabilitation and induce pain in the patient. The rehabilitation makes it possible to reform the tunnel(s) in which the tendon passes forcefully so that the adhesions or deformations pass forcefully in said tunnel(s).

The implantable device according to the invention can be implemented for the repair of at least one tendon by a surgical treatment method comprising the steps of:
providing two tendon portions to be repaired;
optionally, performing a particularly temporary suture on each of the tendon portions to be joined, each suture makes it possible to place said portion in the interior volume of the braid into the interior volume of the central part;
arranging the two tendon portions to be treated in the interior volume of the braid according to the invention so that said two portions overlap and are arranged in the central repair part of the braid; this arrangement resulting in a radial expansion of the central part of the braid;
preferably, arranging a first tendon section at least partially in the interior volume of a first lateral part and arranging a second tendon section at least partially in the interior volume of a second lateral part; the first section comprises a portion to be repaired and the second section comprises a portion to be repaired, said two portions to be repaired being housed, in particular by being superimposed at least partially, in the interior volume of the central part of the braid;
making at least one suture between the first lateral part and the first tendon section in order to assemble the first lateral part, in particular the first free end of the braid, to the tendon, in particular to the first tendon section;
making at least one suture between the second lateral part and the second tendon section in order to assemble the second lateral part, in particular the second free end of the braid, to the tendon, in particular to the second tendon section;
optionally suturing said two partially superimposed portions using at least one suture thread (particularly not necessary for the tendons of the hand but can be useful for other applications).

In one embodiment, the braid has, at rest, a length greater than or equal to about 10 mm and less than or equal to about 40 mm.

In one embodiment, the central part has, at rest, a length greater than or equal to about 5 mm and less than or equal to about 20 mm.

In one embodiment, the first lateral part and/or the second lateral part each has/have, at rest, a length greater than or equal to about 2 mm and less than or equal to about 10 mm.

In one embodiment, the braid has a diameter at rest ranging from 2 mm to 5 mm (the diameters of the central repair part and of the first and second lateral parts are of the same order at rest).

In the present text, it is meant by “at rest” the braid in a position free from any stress aiming at deforming it, which is in particularly empty.

In the present text, it is meant by “braiding angle” the angle formed between two intersecting braided strands or two oblique strands, the braiding angle being open on one of the first and second free ends of the braid. The braiding angles are measured in the same manner and in the same direction in the different parts of the braid. Preferably, the braiding angle β at rest in said at least one region of the central part is greater than 0° and less than or equal to 45°, in particular less than or equal to 30°, preferably less than or equal to 20°.

Preferably, the braiding angle α1 at rest in said at least one region of the first lateral part and/or the braiding angle α2 at rest in said at least one region of the second lateral part at rest is/are greater than or equal to 45° and less than or equal to 90°, in particular greater than or equal to 50°.

The braid is preferably a bi-axial braid.

The braid has preferably a generally cylindrical shape (at rest).

The braid has preferably a longitudinal axis L, particularly extending between its first and second ends, which are in particular free.

The braid has preferably a transverse axis T, which is secant, in particular substantially perpendicular, to the longitudinal axis L.

In one embodiment, the braid comprises through openings, preferably having at least one dimension greater than or equal to 0.2 mm, more preferably greater than or equal to 0.5 mm.

This arrangement promotes the exchange of fluids between the interior volume of the braid and the exterior environment of the braid, thus promoting the repair of the damaged or cut ends.

In one embodiment, the central part and/or the first lateral part and/or the second lateral part and/or the braid is/are completely bioresorbable or partially bioresorbable or non-bioresorbable.

The implantable device according to the invention is configured to be implanted in the body of a human or an animal.

The implantable device according to the invention can be used for the repair of at least one tendon or ligament of the hand, the elbow, the shoulder, the foot, the knee, the hip and the vertebral column, in particular for human or veterinary surgical use.

Preferably, the braid is braided on a braiding machine comprising from 4 to 48 spindles, in particular from 4 to 24 spindles, for example of the order of 24 spindles.

The braid preferably comprises from 4 to 48 strands (particularly each strand being supported by a spindle), more preferably from 4 to 32 strands, for example between 4 to 24 strands, in particular of the order of 24 strands.

Preferably, the braid comprises one or more monofilament thread(s).

Preferably, the braid comprises one or more multifilament thread(s).

Preferably, the braid comprises one or more monofilament thread(s) and one or more multifilament thread(s). This last arrangement promotes the mechanical properties sought for the braid (expansion and elongation of the central part, friction exerted by the lateral parts)

The monofilament and/or multifilament thread(s) can comprise or is/are made up of one or more material(s) chosen from: polyethylene terephthalate, polybutylene terephthalate, polyolefins (for example polypropylene or polyethylene or ultra-high molecular weight polyethylene), polyamides (for example polyamide 6, polyamide 6-6 or 4-6), an L or D or L,D-form lactic acid polymer, a glycolic acid polymer, a lactic acid and glycolic acid copolymer, or a mixture thereof.

In one embodiment, said at least one monofilament thread has an elongation at rupture less than or equal to 100%, particularly less than or equal to 70%, more particularly less than or equal to 60%, and greater than or equal to 15%, in particular greater than or equal to 20%.

In one embodiment, said at least one monofilament thread has a load at rupture greater than or equal to 150 cN, preferably greater than or equal to 200 cN, particularly less than or equal to 500 cN.

In one embodiment, said at least one monofilament thread has a tenacity (cN/dtex) greater than or equal to 1 and less than or equal to 30, preferably less than or equal to 20, more preferably less than or equal to 15, particularly less than or equal to 10.

The load at rupture, the tenacity and the elongation at rupture of the monofilament thread can be measured according to the EN 13392 standard dating from June 2003.

In one embodiment, said at least one monofilament thread has a count (dtex) greater than or equal to 20 dtex and less than or equal to 100 dtex, in particular less than or equal to 80 dtex. The count can be measured using the NF EN 13392 standard dating from September 2001.

In one embodiment, said at least one multifilament thread has an elongation at rupture less than or equal to 100%, particularly less than or equal to 60%, more particularly less than or equal to 30%, and greater than or equal to 5%, in particular greater than or equal to 10%.

In one embodiment, said at least one multifilament thread has a load at rupture greater than or equal to 200 cN, preferably greater than or equal to 500 cN, more preferably greater than or equal to 800 cN, particularly less than or equal to 4,000 cN.

In one embodiment, said at least one multifilament thread has a tenacity (cN/tex) greater than or equal to 10 cN/tex, preferably greater than or equal to 30 cN/tex, more preferably greater than or equal to 60 cN/tex, particularly less than or equal to 200 cN/tex.

The load at rupture, tenacity and elongation at rupture of the multifilament thread can be measured according to the DIN EN ISO 2062 standard dating from January 2010.

In one embodiment, said at least one multifilament thread has a count (dtex) greater than or equal to 20 dtex and less than or equal to 100 dtex, in particular less than or equal to 80 dtex. The count can be measured using the NF EN 13392 standard dating from September 2001.

In one variant, the braid comprises a number of stitches per inch which is gradually decreasing from said at least one region of the first lateral part towards said at least one region of the central part.

Preferably, the braid comprises a number of stitches per inch which is gradually decreasing from said at least one region of the second lateral part towards said at least one region of the central part.

In one variant, the number of stitches per inch is gradually increasing between the central part and the first lateral part on the one hand, and between the central part and the second lateral part on the other hand.

In the present text, it is meant by “stitch” the crossing between two braided strands. One inch corresponds to 25.4 mm.

In one variant, the first lateral part and/or the second lateral part has/have a number of stitches per inch greater than or equal to 10 and less than or equal to 20.

In one variant, the first central part has a number of stitches per inch greater than or equal to 1 and less than or equal to 15.

In one variant, the number of stitches per inch in said region of the central repair part is less than, preferably less than or equal to 1.2, times the number of stitches per inch in said region of the first lateral part.

In one variant, said region of the central repair part comprises a number of stitches per inch which is less than, preferably less than or equal to, 1.2 times the number of stitches per inch in said region of the second lateral part.

In one variant, the braid is structurally configured so that, under the effect of a determined internal deformation, the rate of radial expansion of the first or second lateral part is smaller than the rate of radial expansion of the central part.

Preferably, the rate of radial expansion is taken by measuring the difference in the optical diameter of a braid between a position at rest and a position where the braid is compressed according to a determined force in the direction of its length, particularly stretched according to each of its first and second free ends.

In the present text, it is meant by “determined internal deformation” the deformation obtained for example by placing the tendon to be repaired in the interior volume of the braid, particularly so that a first tendon section is arranged in the interior volume of the first lateral part, so that a second tendon section is arranged in the interior volume of the second lateral part and so that the portions to be repaired of said first and second sections are arranged in the central part, particularly in a superimposed or juxtaposed manner.

Preferably, the central part has a diameter D0 at rest, and is configured to expand radially under the effect of an internal deformation so as to present a diameter De in a determined expanded state greater than the diameter D0.

In one variant, under the effect of a determined internal deformation, the central part has an expanded diameter De greater than or equal to 1.5 times its diameter at rest DO (De≥1.5*D0).

In one variant, the first lateral part has a diameter at rest D1, and is structurally configured to have an expanded diameter D1e under the effect of a determined internal deformation, with D1e less than or equal to 1.5 times D1 (D1e≤1.5*D1).

In one embodiment, the second lateral part has a diameter at rest D2, and is structurally configured to present an expanded diameter D2e under the effect of a determined internal deformation, with D2e less than or equal to 1.5 times D2 (D2e≤1.5*D2).

In one variant, the braid comprises an inner surface with a reference boundary. Said braid further comprises at least one thread, in particular at least one monofilament thread, which projects at regular intervals from said reference boundary into the interior volume of said braid, particularly said at least one thread forms regions in relief (more particularly evenly distributed regions).

The regions in relief are designed to penetrate the outer surface of the tendon housed in the interior volume of the braid without shearing it.

Advantageously, the load exerted by the muscles is supported in part by the sutures and also by the internal roughness of the braid.

In one variant, the braid comprises one or more braided strands, at least one of the strands comprises at least one monofilament thread and at least one multifilament thread.

It has been found that this mixture of a monofilament thread and of a multifilament thread makes it possible to compress the tendon without shearing it.

Said at least one monofilament thread and said at least one multifilament thread have a combined action. In the tendon blocking mechanism, said at least one multifilament thread works in friction with the tendon and said at least one monofilament thread then works by impacting the outer surface of the tendon.

In one variant, said at least one strand comprises at least one monofilament thread and at least one multifilament thread arranged in a substantially parallel manner (that is to say without imparting any particular torsion).

This arrangement allows more exposure of the monofilament thread in the regions in relief.

In one variant, the braid comprises at least one monofilament thread having a diameter less than or equal to 350 μm, particularly greater than or equal to 50 μm, preferably less than or equal to 180 μm.

It may be said thread forming regions in relief defined above and/or arranged in parallel with at least one multifilament thread.

In one variant, the braid comprises at least one multifilament thread comprising at least four filaments, at least one of the filaments of which has a count less than or equal to dtex, in particular less than or equal to 10 dtex.

In one embodiment, said at least one multifilament thread has a count greater than or equal to 50 dtex, particularly less than or equal to 300 dtex.

In one embodiment, said at least one multifilament thread comprises a number of filaments greater than or equal to 4 and less than or equal to 300, particularly less than or equal to 200, more particularly less than or equal to 100, in particular ranging from to 60.

Preferably, said multifilament thread is arranged parallel to said monofilament thread as defined above in at least one strand.

In one variant, the hollow braid has a longitudinal axis L, and the central part has an elongation, in the longitudinal direction under a given load, greater than the elongation of the first lateral part, under said load, in the longitudinal direction, in particular said load is less than or equal to 200 Newtons.

Preferably, the central part has an elongation under a given load, in particular less than or equal to 200 Newtons, in the longitudinal direction, greater than the elongation of the second lateral part, under said load, in the longitudinal direction.

Said load is preferably less than or equal to 200 Newtons for the repair of at least one tendon or ligament of the hand.

This load can reach about 800 Newtons (within +/−100 Newtons) depending on the type of tendon or ligament to be treated.

The more open structure (fewer stitches per inch and smaller braiding angle) allows the central part to deform and to elongate more than the first and second lateral parts. Thus, the superimposed portions to be repaired slide as the central part elongates under the effect of the tensile forces exerted by the muscles on the tendons.

In one variant, the central part in an expanded state has a length (Lch) smaller than its length (L0) at rest, and under the effect of a given longitudinal load, in particular less than or equal to 200 Newtons, the central part elongates to reach a length (La) smaller than or equal to the length (L0) at rest of the central part.

The measurement is performed by optically measuring the angle variation of the structure by observing a thread of the braid of a different color.

The measurement can also be performed on a traction bench, a first free end of the braid is held in the jaws in a fixed manner, while the second free end of the braid, also held in the jaws, moves.

In one variant, the length of the central part is greater, particularly by at least 1.5 times, than the length of the first lateral part.

In one embodiment, the length of the central part is greater, particularly by at least 1.5 times, than the length of the second lateral part.

In one embodiment, the first lateral portion has a length of the order of the length of the second lateral part.

### DESCRIPTION OF THE DRAWINGS

The invention will be better understood upon reading the following description of one embodiment of the invention given by way of non-limiting example, with reference to the appended drawings, in which:

FIG. 1 schematically illustrates the distribution of the tendons of the hand according to five areas I-V;

FIG. 2 schematically illustrates the arrangement of an exemplary tendon of the hand passing through different tendon sheaths in connection with the bones of the hand;

FIG. 3 schematically illustrates, by way of illustration and without limitation, different suture techniques of the state of the art;

FIG. 4 schematically represents a first exemplary implantable device according to the invention at rest;

FIG. 5 schematically represents the first exemplary implantable device of FIG. 4, with the central part in an expanded state in which the tendon portions overlap over the entire length of the central repair part;

FIG. 6 schematically represents the first exemplary implantable device of FIG. 4, with the central part in an expanded state in which the tendon portions overlap over part of the length of the central repair part;

FIG. 7 schematically represents the first exemplary implantable device of FIGS. 4 and 5 or 6 at the end of the fusion of the tendon portions to be repaired;

FIG. 8 schematically represents the crossing of the strands of the braid of FIG. 4;

FIG. 9 schematically represents the crossing of the strands of the braid of FIG. 5 or 6.

### DESCRIPTION OF THE EMBODIMENTS

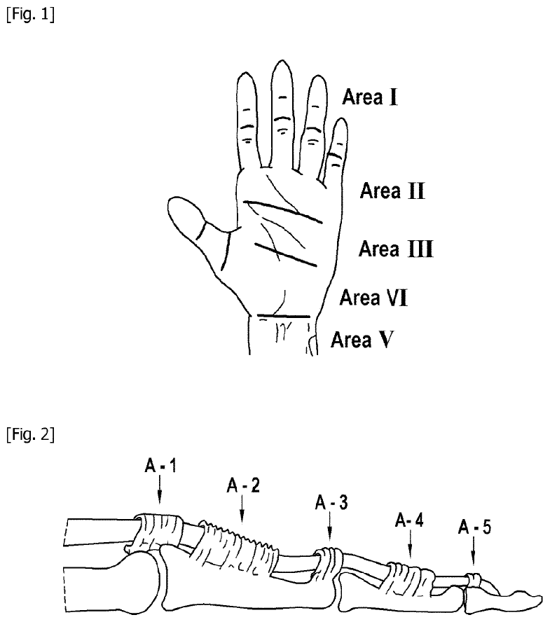

FIG. 1 schematically represents the different tendons of the hand according to the reference anatomical classification adopted in 1980 by Verdan and Michon. This classification divides the wrist and the hand into distinct areas, each characterizing the position of the two flexor digitorum profundus (FDP) and flexor digitorum superficialis (FDS) tendons therebetween, and their relation with the adjacent structures. These areas at the level of the long fingers are five in number. The area 1 (ZI) is located downstream of the proximal interphalangeal from which there is only one tendon left in the digital sheath. This area is characterized by the proximity of the insertion of the tendon, source of vascularization and the possibility of performing a solid suture. The area 2 (ZII), also called Bunnell's “no man's land” area, is characterized by a narrow osteo-fibrous duct, tight flexors and precarious vascularization. The areas 3, 4, 5 (ZIII, ZIV, and ZV) are characterized by good tendon vascularization and a much freer tendon travel. This classification also applies to the thumb where there is only one tendon, the flexor pollicis longus (FPL) whose vascularization is better.

The area 2 (ZII) is particularly delicate for the repair of the flexor tendon because: 1/ two distinct flexor tendons are superimposed at this level: the flexor digitorum profundus and the flexor digitorum superficialis; 2/ the tendon holding pulleys are narrow; 3/ the repair of the tendons is difficult because the cut tendons tend to retract, especially the proximal end; it is sometimes necessary to reach for them several centimeters away, by flexing the finger and the wrist, to recover the healthy tendinous ends; 4/ the vascularization of the tendons is segmental and ensured by perforating mini-arteries; these can be torn off during the initial trauma, which will complicate the internal tendon healing after the repair; 5/ the tendon suturing technique varies according to each surgeon and his habits; 6/ the early post-operative rehabilitation cannot be an absolute dogma, because it is necessary to take into account the psychology of the patient who is more or less attentive to his body, and more or less brutal in his daily gestures.

FIG. 2 illustrates a tendon of the hand passing through different fibrous sheaths referenced A1 to A5.

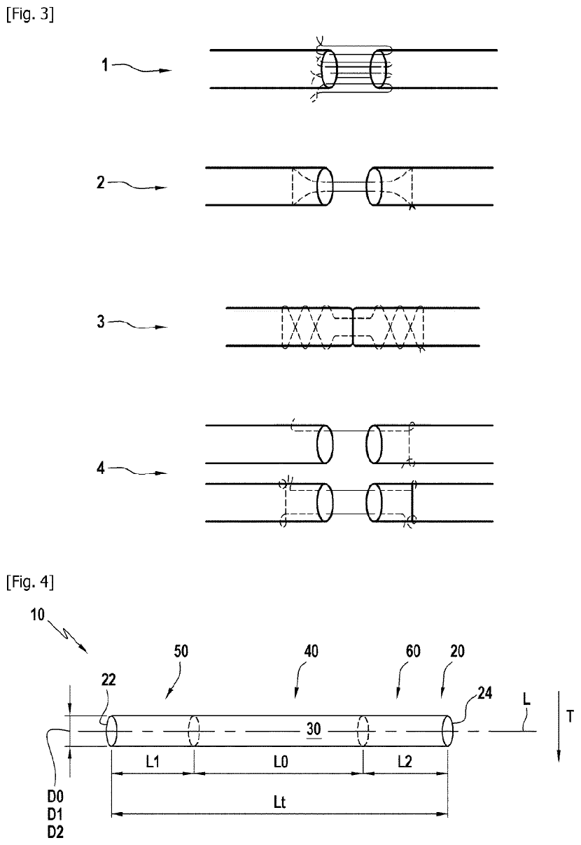

FIG. 3 schematically represents various suturing techniques illustrating part of the suturing techniques used in the state of the art by way of illustration and without limitation to secure the ends of the cut tendons. The first variant 1 of FIG. 3 comprises 4 interrupted sutures, the second variant 2 is called Nicoladoni technique, the third variant 2 is called Bunnell suture, and the fourth variant 4 is called Kessler technique (represented in two steps).

A first example of implantable device 10 is represented in FIG. 4 in an unexpanded state, at rest. This device 10 comprises a hollow braid 20 having an interior volume 30 and comprising over its length a central repair part 40 arranged between first and second lateral parts 50, 60. The braid 20 has in this specific example a generally tubular shape at rest (that is to say free of any stress tending to deform it). The braid 20 has a longitudinal axis L extending between its first and second free ends 22, 24, particularly passing substantially through the center of the braid 20. The braid 20 has a transverse axis T substantially perpendicular to the longitudinal axis L. The braid 20 has a total length Lt, for example of the order of 22 mm. The central part 40 has a length L0, for example of the order of 12 mm. The first and second lateral parts 50, 60 have lengths respectively L1 and L2, in particular substantially of the same order, for example of the order of 5 mm. The lengths L1 and L2 can be different depending on the dimensions of the tendon to be repaired.

The braiding angle varies between the central part 40 and the first lateral part 50 on the one hand, and between the central part 40 and the second lateral part 60 on the other hand. Particularly, the braiding angle gradually decreases from the first lateral part 50 towards the central part 40 to gradually increase from the central part 40 towards the second lateral part 60.

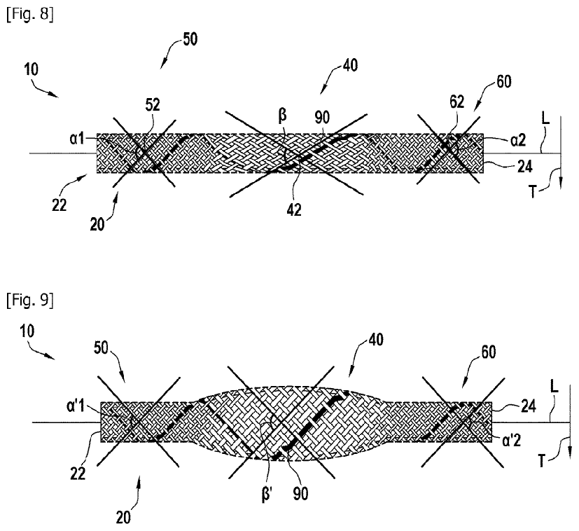

The braiding angle β, represented in FIG. 8, in at least one region 42 of the central part 40, is comprised between 0° and 90°, particularly less than 45°.

The braiding angle α1, represented in FIG. 8, in at least one region 52 of the first lateral part, is comprised between 45° and 90°.

The braiding angle α2, represented in FIG. 8, in at least one region 62 of the second lateral part, is comprised between 45° and 90°.

The braiding angle α1 is of the same order as the braiding angle α2. These braiding angles could be different as long as they remain greater than β in order to preserve the targeted mechanical properties.

The braiding angles, to be able to be compared, are measured in the same direction and in the same way. The braiding angles β, α1 and α2 are open on one of the first and second ends 22, 24 of the braid 20.

The braiding angle β is thus smaller than the angle α1, and the angle α2.

In one embodiment, the braid 20 is a bi-axial braid, braided on a 24-spindle braiding machine, each spindle supporting a braiding strand. A strand comprises at least one monofilament thread and at least one multifilament thread arranged in parallel. Preferably, the monofilament thread has a diameter comprised between 0.05 mm and 0.10 mm. The thread is preferably polypropylene. The multifilament thread comprises at least 4 filaments, preferably between 40 and 60 filaments. The multifilament thread has preferably a count comprised between 150 dtex and 210 dtex. The multifilament thread is preferably made of polyethylene terephthalate.

As represented in FIG. 4, the central part 40 has a diameter D0 at rest, the first lateral part has a diameter D1 at rest, and the second lateral part has a diameter D2 at rest. In this specific example, D0, D1 and D2 are of the same order, for example about 3.75 mm.

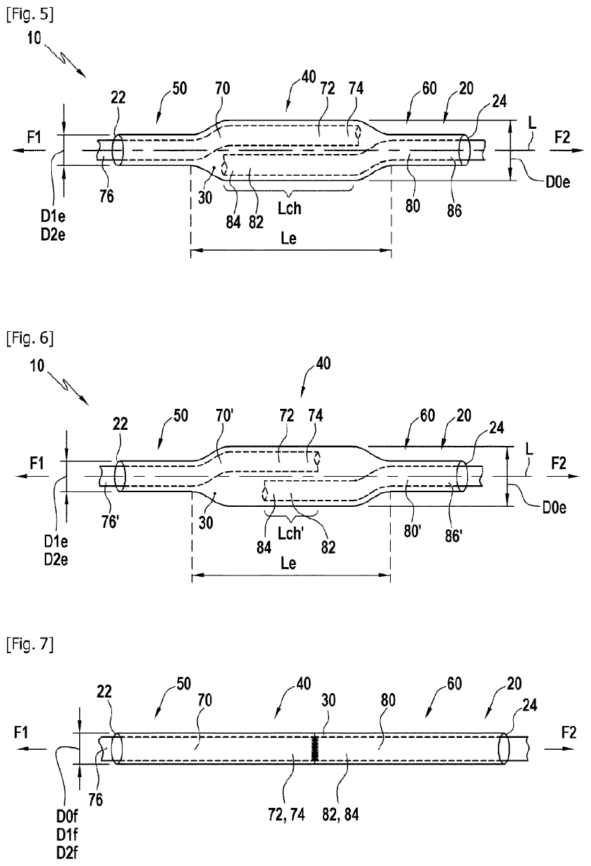

In operation, as represented in FIG. 5, a first tendon section 70 to be repaired is introduced into the interior volume 30 of the first lateral part 50 and a second tendon section 80 to be repaired is introduced into the interior volume 30 of the second lateral part 60. The first tendon section 70 comprises a proximal portion to be repaired 72 arranged at the level of its first end 74, and a distal portion 76 sutured to the first free end 22 of the braid 20. The second tendon section 80 comprises a proximal portion to be repaired 82, arranged along its first end 84, and a distal portion 86 sutured to the second free end 24 of the braid 20. The sutures are not represented. The sutures can be performed using a suture thread passing between the stitches of the braid 20 and through the first or second tendon section, according to a trajectory determined by the practitioner. The proximal portions to be repaired 82 and 72 are preferably free, or optionally partially joined by means of a suture, and are arranged by partially overlapping or butted in the central part 40. In this specific example, the proximal portions 82 and 72 overlap over an overlap length Lch, represented as being substantially equal to the length Le in an expanded state of the central part 40. However, the overlap length of the proximal portions 72 and 82 may be smaller than the expanded length Le of the central part 40 as represented in FIG. 6 with Lch′. Lch′ or Lch can be for example of the order of 3-4 mm. The central part 40 is in an expanded state in FIG. 5 or 6 and 9. This state is obtained due to the internal deformation applied by the particularly overlapping joined portions 72 and 82. The expanded diameter of the central part 40 is thus DOe, for example of the order of 1.5 times DO, for example about 5.63 mm. The diameters of the first and second lateral parts 50 and 60 are respectively D1e and D2e, and are in this specific example of the order of D1 and D2. The first and second lateral parts can expand but very little, they are not configured to expand significantly unlike the central part 40. The braid 20 comprises a strand 90 represented in black in FIGS. 8 and 9. This strand 90 forms a helix whose pitch varies between the central part 40 and the first and second lateral parts 50, 60. It is observed that the pitch of the helix formed by the strand 90 in the central part 40 is reduced when the central part 40 passes from a state at rest in FIG. 8 to an expanded state in FIG. 9. The braiding angle β′ increases in FIG. 9, since the strands tend to move apart, and is therefore greater than β. The angles α1 and α2 can be of the same order as α1′ and α2′, or greater.

FIGS. 5 and 6 schematically represent two variants of arrangement of tendon sections for the repair of said tendon after surgical intervention but before healing.

Under the effect of rehabilitation, the first and second tendon sections 70 and 80 are tensioned according to the opposite arrows F1 and F2 represented in FIGS. 5 to 7, said sections 70, 80 being in connection with the braid 20, the latter elongates. The applied forces F1 and F2 are for example less than or equal to 200 Newtons. The central part 40 is thus configured so that its elongation is controlled so that the portions to be repaired 72 and 82 remain in contact long enough for them to fuse. The central part 40 can thus elongate of the order of the length of the overlap, namely of the order of Lch, or over a lesser length, in particular to reach its initial length L0 at rest. For example, this elongation is of the order of 30%. The first and second lateral parts 50 and 60 can also elongate but less than the central part 40. When the tensile forces F1 and/or F2 is/are applied, the first and second lateral parts 50 and 60 apply a pre-tension on the tendon sections 70 and 80 making it possible to initiate frictional forces with said sections, which promotes the contact with the tendons while the central part 40 does not exert such a frictional and compressive effect. The central part 40 being expandable, it is not necessary for the portions to be repaired 72 and 82 be cut cleanly, they can still be in connection and thus form a damaged/degenerated area to be repaired and not ruptured or cut.

At the end of the sliding of the portions 72 and 82, these are substantially juxtaposed and fused, as illustrated in FIG. 7. The central part 40 thus has a diameter D0f, which can be of the order of DO, or significantly lower. The first and second lateral parts 50 and 60 have diameters at the end of the fusion, as represented in FIG. 7, D1f and D2f, which can be of the same order or substantially smaller than the diameters D1 and D2 at rest.

The device 10 according to the invention was tested in comparison with a suture called Pulvertaft suture 3 (3 weaves and 10 interrupted sutures) on sheep flexor tendons (ranges of 24 tendons per tested device). The tested braid has a length Lt of 50 mm and a diameter D0 (of the order of D1 and D2) of 3.75 mm at rest. The distal portions of the first and second sections in connection with the first and second free ends of the braid are sutured by using a continuous suture called interlocked circular suture. The suture thread used has as reference: 4-0 Prolene® Polypropylene Suture. The tendons treated with the Pulvertaft suture are the tendons said of the control group. The biomechanical tests have been performed with a hydraulic tensile testing machine (LRX Plus with 250N load cell, Lloyd Instruments, Bognor Regis, UK) by using custom-made forceps with serrated teeth. Each tendon is secured in the upper and lower forceps with sandpaper to prevent the tendons from sliding. The length between the upper and lower forceps is standardized at 50 mm. During a cycle test, the tendon sections to be repaired are kept moist by spraying a saline solution on them. The sutured tendons are pre-stretched with 2N for 50 seconds before being tested. Then, 500 cycles of mechanical load between 2N and 15N are applied at a frequency of 1 Hz. The tendons are each time relaxed at a speed of 20 mm/s until complete rupture.

The average length and the average surface CAS of the area to be repaired are measured: for the tendons studied according to the invention and for those studied in the control group.

Before repair, the average CSA for the invention (the central part of the braid comprising the at least partially superimposed portions to be repaired) is of 11.77 mm² (SD 1.22) and of 11.63 mm² (SD 2.08) for the control group.

After repair, the average length of the central part is of 14.77 mm (SD 1.59) compared to 37.21 mm (SD 2.77) for the control group, and the average CSA is of 27, 82 mm² (SD 7.31) for the invention versus 34.47 mm² (SD 4.48) for the control group.

After 500 cycles, no rupture or hole formation is observed for the invention and the control group.

The mechanical properties recorded are as follows:
1/ the average ultimate load (N) for the invention is of 204.08 (SD 21.41), versus 167.35 (SD 27.59) for the control group;
2/ the stress at the average ultimate load (MPa) is of 17.57 (SD 2.63) for the invention versus 14.61 (SD 2.53) for the control group;
3/ the extension of the preload under the ultimate load (mm) is of: 24.35 (SD 3.12) for the invention versus 17.35 (SD 2.85) for the control group;
4/ the rigidity (N/mm) is of: 17.41 (SD 2.92) for the invention versus 21.83 (SD 2.87) for the control group.

It has thus been observed that a repair is possible with the device according to the present invention with a junction area of the possibly superimposed portions to be repaired of only about 15 mm, whereas a Pulvercraft suture requires a length of the repair area of almost 4 cm. The present invention thus offers the possibility of repairing restricted tendon areas, and offers more possibilities for the tendon transfer surgery. It is also noted that the final CSA is smaller with the device according to the invention, which means that the repaired area is less bulky. This arrangement also facilitates the displacement of the repaired tendon into the fibrous sheaths.

The implantable device 10 according to the invention was also tested in comparison with a suture called modified multi-strand Kessler suture with a 4-0 Prolene® loop and a Silfverskiöld repair using a 6-0 Ethilon® suture on sheep flexor tendons (ranges of 46 tendons per tested device). The control group corresponds to the results associated with the comparative suture. The tested braid has a length Lt of 18 mm and a diameter DO (of the order of D1 and D2) of 3 mm at rest. The distal portions of the first and second tendon sections in connection with the first and second free ends of the braid are sutured by using a continuous suture called interlocked circular suture. The suture thread used has as reference: 4-0 Prolene® Polypropylene Suture.

Immediately after the repair, the tendons are placed in the test machine defined above. The repaired tendons according to the invention and the tendons of the control group undergo a cyclic load until rupture for a first group of 23 tendons and a static load until rupture for a second group of 23 tendons. Each tendon is subjected to a preload of 1N for the static and cyclic tests.

For the test of the static load at rupture, the tendons are elongated until rupture at a speed of 20 mm/s after pre-tension.

The cyclic test is performed with an incremental load protocol. Every 500 cycles, the load is increased (2-20N; 3.3-33 N; 4.5-45 N; 6-60 N; 7.5-75 N; 9-90 N and finally 10.5-105 N). Every 100 cycles, the formation of holes of 1 mm, 2 mm or 3 mm or a rupture is observed.

Before repair, the average CSA of the tendons for the invention is of 8.43 mm² (SD 1.46) and 8.38 mm² (SD 2.02) for the control group.

The results of the biomechanical tests are as follows:
average ultimate load (N): 98.27 (SD 12.66) for the invention and 62.99 (SD 11.08) for the control group;
the average load stress (MPa): 11.78 (SD 1.17) for the invention and 8.09 (SD 3.13) for the control group;
the average rigidity (N/mm): 7.09 (SD 2.9) for the invention and 8.76 (2.15) for the control group;
opening of 1 mm, average ultimate load (N): 78.49 (SD 6.17) for the invention and 54.18 (SD 14.61) for the control group;
2 mm opening, average ultimate load (N): 82.26 (SD 6.03) for the invention and 51.74 (SD 13.86) for the control group;
3 mm opening, average ultimate load (N): 86.02 (SD 7.69) for the invention and 50.1 (SD 11.61) for the control group.

Different types of rupture are observed for the two repair techniques according to the static test. 70% of the tendons repaired according to the invention rupture due to gradual tear. The remaining 30% show a rupture of the circular interlock suture connecting the proximal portions of the tendon sections to the ends of the braid. The ultimate rupture always takes place after the appearance of the 3 mm opening.

The repaired tendons of the control group rupture in 100% of the cases due to a rupture of the suture joining the portions to be repaired. In 17 cases out of 23, the rupture takes place for the control group before the appearance of the 3 mm opening. The braid according to the invention thus offers a more solid repair both in the static test and in the dynamic test, which would allow immediate mobilization with a safety margin.

When comparing the results of the invention and of the control group, the two techniques should make it possible to authorize immediate mobilization estimated as requiring a resistance at rupture between 35 N and 45 N.

However, based on the results of the cyclic tests, the tendons repaired according to the invention begin to rupture from 1,700 cycles whereas for the control group, the last surviving tendons rupture at 1,800 cycles. In the control group, 50% of the tendons show the formation of a 3 mm opening or a complete rupture during the 4.5-45 N cycles. In comparison, 50% of the tendons repaired with the braid of the invention show a 3 mm opening during the 7.5-75 N cycles, and only at 9.5-95 N, 50% of the tendons repaired according to the invention rupture.

The device according to the invention thus offers significant resistance allowing immediate mobilization with a significant safety margin.

1. An implantable device for repairing at least one tendon or ligament wherein it comprises a hollow braid configured to receive in its interior volume at least one portion of a tendon or ligament and comprising over its length a central repair part arranged between first and second lateral parts, in that the braiding angle in at least one region of the central part at rest is smaller than the braiding angle in at least one region of the first lateral part at rest and/or smaller than the braiding angle in at least one region of the second lateral part at rest.

2. The implantable device according to claim 1, wherein the braid comprises a number of stitches per inch which is gradually decreasing from said at least one region of the first lateral part towards said at least one region of the central part.

3. The implantable device according to claim 1, wherein said region of the central repair part comprises a number of stitches per inch which is less than, preferably less than or equal to, 1.2 times the number of stitches per inch in said region of the first lateral part.

4. The implantable device according to claim 1, wherein the braid is structurally configured so that, under the effect of a determined internal deformation, the rate of radial expansion of the first or second lateral part is smaller than the rate of radial expansion of the central part.

5. The implantable device according to claim 1, wherein under the effect of a determined internal deformation, the central part has an expanded diameter D0e greater than or equal to 1.5 times its diameter at rest D0.

6. The implantable device according to claim 1, characterized in that the first lateral part has a diameter at rest D1, and is structurally configured to have an expanded diameter D1e under the effect of a determined internal deformation, with D1e less than or equal to 1.5 times D1.

7. The implantable device according to claim 1, wherein the braid comprises an inner surface with a reference boundary, and wherein it comprises at least one thread, which projects at regular intervals from said reference boundary into the interior volume of said braid.

8. The implantable device according to claim 1, wherein the braid comprises one or more braided strands, at least one of the strands comprises at least one monofilament thread and at least one multifilament thread.

9. The implantable device according to claim 8, wherein said at least one monofilament thread and said at least one multifilament thread are arranged in a substantially parallel manner.

10. The implantable device according to claim 1, wherein the braid comprises at least one monofilament thread having a diameter less than or equal to 350 μm.

11. The implantable device according to claim 1, wherein the braid comprises at least one multifilament thread comprising at least four filaments, at least one of the filaments of which has a count less than or equal to 15 dtex.

12. The implantable device according to claim 1, wherein the hollow braid has a longitudinal axis L, wherein the central part has an elongation, in the longitudinal direction L under a given load, greater than the elongation of the first lateral part under said load and in said longitudinal direction.

13. The implantable device according to claim 1, wherein the central part, in an expanded state, has a length Lch smaller than its length L0 at rest, and under the effect of a given longitudinal load, the central part elongates to reach a length La smaller than or equal to the length L0 at rest of the central part 40.

14. The implantable device according to claim 1, wherein the length of the central part is greater than or equal to, the length of the first lateral part.

15. The implantable device according to claim 3, wherein the number of stitches per inch is less than or equal to, 1.2 times the number of stitches per inch in said region of the first lateral part.

16. The implantable device according to claim 7, wherein said at least one thread is a monofilament thread.

17. The implantable device according to claim 7, wherein said at least one thread forms regions in relief.

18. The implantable device according to claim 10, wherein the at least one monofilament thread has a diameter greater than or equal to 50 μm.

19. The implantable device according to claim 11, wherein at least one of the filaments of which has a count less than or equal to 10 dtex.

20. The implantable device according to claim 14, wherein the length of the central part is greater or equal by at least 1.5 times the length of the first lateral part.
